(19) United States
(12) Patent Application Publication
(10) Pub. No.: US 2010/0137256 A1
(43) Pub. Date: Jun. 3, 2010

(54) Device and Method for Treating Human Body

(76) Inventor: Colette Haddad, Toronto (CA)

Correspondence Address: Nasser Ashgriz Heydary Hamilton PC;Suite 1200, 439 University Ave., Toronto, ON, M5G 1Y8, CA

(21) Appl. No.: 12/324,915

(22) Filed: Nov. 28, 2008

(51) Int. Cl. A61M 1/00; A61B 18/04; A61K 31/685; A61P 43/00

(52) U.S. Cl. 514/114; 604/315; 606/27

(57) ABSTRACT

A method for contouring the body by destroying adipose tissue is disclosed. This method can reduce the circumference of the treated area, improve skin contour irregularities and smooth the appearance of the skin surface. The method is also used as a body treatment before and after a liposuction procedure.

### FIELD OF THE INVENTION

The present invention relates to a method and a system to enable therapists to perform a non-invasive therapeutics manipulation of body tissues by vacuum-suction.

### BACKGROUND OF THE INVENTION

The lymphatic system is a loosely organized system of vessels and ducts that convey the products of digestion toward the heart. It provides a one-way route for fluid from the tissue spaces between the cells to enter the blood into tiny, capillary-like vessels. The lymphatic system has no pump, so outside forces such as the contraction of muscles are needed to propel the lymph. The lymphatic system is a continuous-cleansing system that operates at the cellular level. Waste products, toxins, and other debris are filtered from the tissues by passing through the lymph nodes. Lymph nodes, found throughout the lymphatic system, are small oval bodies that filter the lymph. When a node is congested, drainage of the lymph is sluggish. Bacteria, red blood cells, infections, and molecules of fat are all things that cause congestion. Lymph transports fats, regulates water flow, eliminates waste matter, and circulates lymphocytes, the white blood cells that participate in immunity. Much of the lymph collects in a large cavity behind the heart called the thoracic duct. Substances from the gastrointestinal tract enter the lymph vessels prior to the bloodstream. The heart then pumps the nutrients to all the body's cells. The cells remove the nutrients they need leaving the liver with the remnants. The lymphatic system, because it carries fat molecules, is the system we work with to reduce congestion. Once mobilized, fat molecules are carried via the lymphatic system to the nearest lymph node. From the lymph node, the fat-saturated lymph is then carried to the thoracic duct where it is transferred to the bloodstream in order to travel to the liver.

Lymphatic drainage promotes a quicker elimination of the toxins and harmful deposits that poison the tissues. The drainage stroke stimulates the lymph flow, reduces blockages of the lymph nodes, and accelerates the metabolism. The factors affecting lymphatic drainage include: A build-up of fat due to poor diet; bacterial infection causing fluid retention; toxins that are stored in our fat cells and are transported by the lymphatic system (some of these toxins are controllable such as preservatives, additives, and chemicals in foods and some of these toxins are out of our control such as environmental pollution); and congested fat, which blocks the passage of the lymphatic system by compressing the lymphatic vessels, causing poor drainage.

Fluid retention slows down lymphatic drainage considerably by causing a low-grade bloating effect. As fat cells enlarge with fluid, what is called osmotic pressure is put on lymph vessels. Ideally there should be a negative osmotic pressure, that is, a slight vacuum. In the spaces between the cells this pressure would “suck” the cells tightly together. Under these circumstances, lymph flow is maximized. When fluid is retained, there is positive osmotic pressure: cells are forced apart, putting pressure on the lymph vessels, thereby reducing lymph flow.

Weight loss is minimal when lymph flow is diminished because fat molecules cannot follow the pathway efficiently. Areas that lose fat easily are those with the best lymphatic drainage. Congestion is always accompanied by poor lymph flow.

The therapeutic goal of the present invention, referred to as Celluderm Technology, is to decrease the size of fat cells and to enhance the lymphatic system by retexturing and smoothing connective-tissue layers subcutaneously. This is beneficial not only on an esthetic level, but also on a therapeutic level to promote lymphatic flow, which leads to a number of health benefits. Celluderm Technology treatments target the lymphatic system and the fat cells in the subcutaneous tissue just below the surface of the skin. The treatments break up the mass of adhesion deposits in the connective tissue, which frees up the muscles, but does not affect muscle tone.

### SUMMARY OF THE INVENTION

The present invention is a non-invasive, portable, and inexpensive system which utilizes a wide range of vacuum suction on different parts of a body to stimulate and improve the lymphatic system; reduce congestion and irregularities; reshape the body in localized areas; reduce water retention; stimulate the immune system; eliminate toxins; enhance elasticity of the skin tissues; and provide relief of muscular pain.

Another object of the present system and method is the stimulation of the lymphatic system in conjunction with liposuction pre-op and post-op to: reduce water retention; speed healing, decrease recovery time, decrease post-op effects by stimulating lymph drainage; reduce irregularities and the bumpy appearance that can occur as a side effect of liposuction; and reshapes the body promoting smoother-looking skin. These and other objects of the present invention will become readily apparent upon further review of the following specification and drawings.

### BRIEF DESCRIPTION OF THE DRAWINGS

In the figures, which illustrate, by way of example only, embodiments of the present invention,

FIG. 1 illustrates a body treatment system comprising of a vacuum system, a filter, a suction cup and a connecting tubing;

FIG. 2 illustrates three different suction cups; and

FIG. 3 illustrates a suction cup with massage rollers.

### DETAILED DESCRIPTION OF THE INVENTION

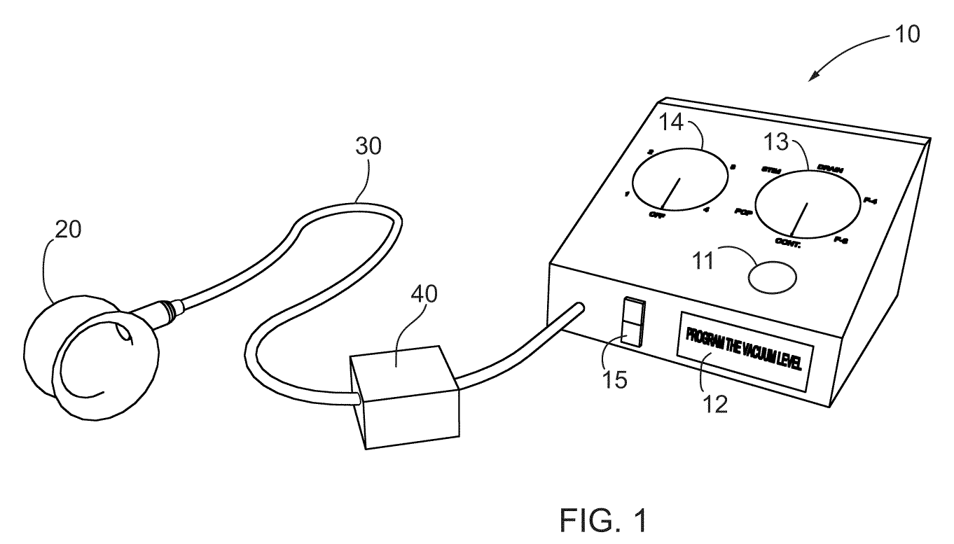

A device for the treatment of human body is illustrated in FIG. 1. This device comprises of a vacuum system 10 and a suction cup 20 connected to the vacuum system through a flexible tubing 30. The vacuum pump assembly comprises of a vacuum pump (not shown), and a gauge that measures the inverse pressure 11, a filter 40 to prevent oil, dust and unwanted material to enter the vacuum pump, a solenoid valve (not shown), and a microprocessor (not shown), which controls the operation of the vacuum pump.

The microprocessor is programmed to provide certain vacuum levels for certain periods for each types of massage. The level of drainage required can be set on the microprocessor, which will adjust the vacuum pump operation accordingly. The microprocessor has a control panel 12 for programming the operation of the solenoid valve and, therefore, the level and duration of the vacuum. Two knobs 13, and 14 on the system provide preset vacuum level and durations, respectively. The following settings are provided on the vacuum system:

1—Facial: This level is used for facial treatments, having a vacuum range from 0-4 inches of mercury. 2—Superficial level: This level is used for arms, calves, shins and forearms, having a vacuum range from 5-10 inches of mercury. 3—Deep Drainage: This level is used for legs, back and stomach having a vacuum range from 10-20 inches of mercury. 4—Deeper Drainage: This level is used for legs, back and stomach, having a vacuum ranges from 20-25 inches of mercury. All pressure units are in inverse pressures.

The machine can also be set at different operating modes for controlling the length of the vacuum strokes. The microprocessor controls the opening and closing of a solenoid valve to provide different operating modes. In each operating mode or scheme, there is a vacuum increasing phase, a main vacuum phase, and a vacuum decreasing phase. Several operating modes are programmed on the systems. These modes are characterized by the duration of the vacuum during the main phase and they include: (1) Pop—for opening of lymph nodes having ½ second duration; (2) Stimulation for stimulating the colon having 1 second duration; (3) Drainage for drainage strokes having 2 second duration; (4) Friction-4 for friction strokes having 4 second duration and (5) Friction-6 for friction strokes having 6 second duration. A knob on the vacuum system is used to set the operating mode.

### The Glass Cups

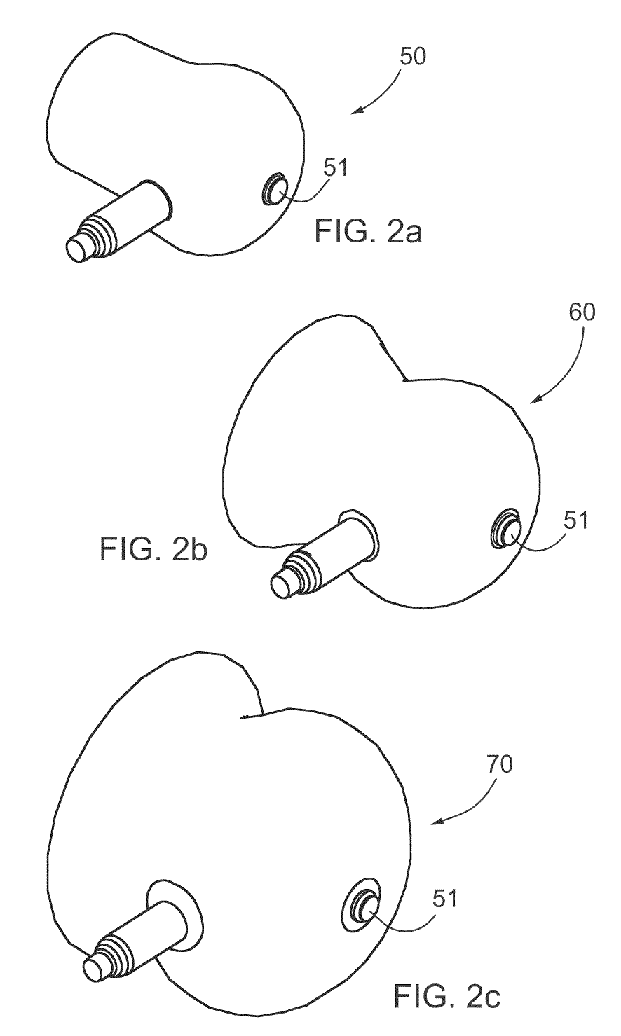

At least three different glass cups are used: a small cup, a large cup, and a face cup as illustrated in FIG. 2. The size of each cup is based on the diameter of the part of the cup that contacts the body. The face cup 50 (FIG. 2a) has a diameter of about ⅞ of an inch; the small cup 60 (FIG. 2b) has a diameter of about 1.5 inches, and the large cup 70 (FIG. 2c) has a diameter of about 2 inches.

Conventional suction cups can be used in this therapy. However, a glass cups illustrated in FIG. 2 are especially designed to allow for the adjustment of the suction through the cup itself using a valve 51 located on the cup. This cup makes it easy to adjust the vacuum during the therapy to reach to a level that the vacuum is effective yet comfortable for the client.

Each cup may also come with a filter (not shown) located on the exit of the cup to block the oil from traveling to the machine.

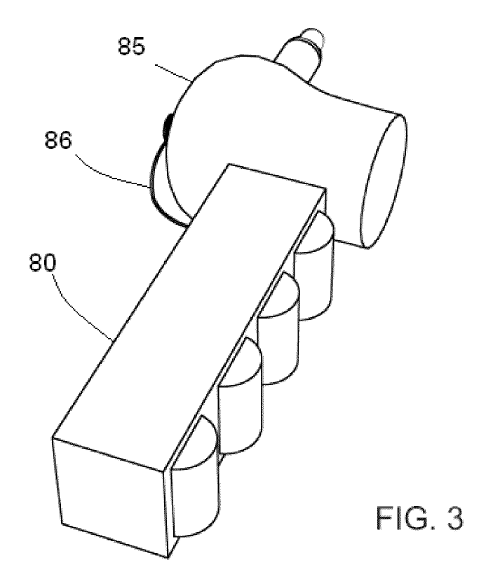

Another embodiment of a cup disclosed here is illustrated in FIG. 3. These cups have set of rollers 80 attached to them. The roller provides a light massage following the suction stroke.

### Method

The method provided here for treating the body and reducing the fat in the body has three main steps: (1) Heating the body, (2) vacuum suction; (3) 24 hours fat free diet.

Prior to applying suction, the fat cells must be heated, which renders the cell membrane more permeable, and allows stored fat to be released more readily. Heat applied to the adipose tissues causes lipolysis by rendering the cell membranes more permeable to allow stored fat to be released. It also shrinks the fat chambers and increases peripheral circulation.

When suction is used on the heated fat cells, fat molecules are then pulled through the cell membrane and released into the lymph fluid surrounding the cell. This reduces the size of the fat cells. It also increases the lymph flow and releases toxins from the fat cells into the lymphatic system.

Vacuum Suction physically breaks fat cell clusters and allows fat molecules to be pulled across the cell membrane and released into the lymph fluid. The application of negative pressure from the vacuum suction dilates the blood vessels and improves local circulation to the skin, enhances lymphatic drainage and stimulates the evacuation of wastes and fat decay products.

And the 24 hours fat-free diet after the treatment allows the fat that was mobilized by the treatment to be used by the body as calories for normal metabolic functions. If additional fat enters the digestive system following the treatment, the fatty acids cannot all be used and some fat will redeposit in the body.

Another component, although not key to the treatment is the consumption of Lecithin. During lipolysis, fat is broken down into free fatty acids which are released into the bloodstream. The fatty acids are relatively hydrophobic. Lecithin is a fat emulsifier that binds fat to water so it can be passed through the body.

### Vacuum Massage

The level of vacuum applied on the body is an important part of the treatment method. The vacuum system utilized in the present method provides a wide range of suctions, preferably between 5-25 inches of mercury (about 2.5-12.5 psi of negative pressure). This allows for treating many layers of the body from superficial drainage to deep drainage. For facial treatments, low levels of vacuum, such as 5 inches of mercury, is preferred. This allows for very light drainage to reduce water retention and stimulate the lymph in the face. For superficial layers of the body, vacuum levels between 5-10 inches of mercury are preferred. For this purpose first a small cup is used to work lightly on the body to enhance lymphatic drainage. This is also a good place to begin if working on the calves, shins, or forearms. For deeper layers of the body, 10-20 inches of mercury vacuums are preferred. This is preferred when working with the large cup. At this level of vacuum, a relatively strong vacuum level is felt on the body, which can breakdown congestion. At even higher levels of vacuum, 20-25 inches of mercury, even deeper layers can be treated, draining the lymphatic system at lower levels. This level of vacuum provides results faster, easier and effortlessly.

The method for treating a person is by first starting the person out at the lowest level needed for the person to feel the suction. Increase the amount of suction with each treatment until the person reaches a happy medium where the treatment is tolerable but he or she can feel something being done.

For this purpose, the vacuum system is equipped with a programmable system that can set the vacuum level as a function of time. There are several set programs, including a gradual increase from a minimal vacuum to a set value at which point it remains constant.

When the person no longer feels any discomfort, the large cup should be introduced. At this point, it may be necessary to turn the machine down to around 8-10 inches of mercury. The large cup can get to deeper layers of the body stimulating the lymphatic system at different levels. Again, the suction is increased in each treatment until the person reaches a stage where there is very little congestion.

The small cup: The small cup is used for superficial layers. Because the mouth of the cup is small, the depth of the treatment is limited. Only the drainage of the superficial lymph vessels improves with the small cup, but there will be visible changes.

Procedure for using the small cup: The strokes should be fast in the beginning, to minimize any pain felt by the person. The movement should always be in the same direction as the flow of the lymphatic system. As the client progresses through the treatments, friction should be used. The long drainage strokes of the friction increases circulation to clear the passageway of the lymphatic system for enhanced drainage. With the proper technique, progress is visible after a few treatments. The hard, lumpy top layer becomes soft and smooth.

When working lengthwise on the muscle, one should always work in an upward direction toward the heart. One should start with a low setting and gradually increase the suction during the progression of treatments, continually consulting with the person to determine the ideal pressure: the point where the person can feel the pressure, but it is still tolerable. To start drainage, one should begin with fast strokes in the direction of the lymphatic system; and slowing down when the person is ready for deeper drainage. One should not use friction during the first treatment.

The large cup: At some point in the treatment, a person will reach a point where his or her progress seems to stop. Despite continuing the treatments and following the fat-free diet, there is no visible change. The next step is to work with the deep fascia, separating the adhesion between individual muscle fibers. The adhesion restricts movements and inhibits lymphatic flow. It is possible to have excellent drainage in the superficial lymph vessels and poor drainage in the deeper lymphatic system. In order to reach the deeper tissue, it is necessary to pick up more flesh in the cup. The bigger mouth on the large cup aids in working with these underlying layers. When working with the large cup, you must alter your technique. If you move quickly when using the large cup, you will only pick up superficial tissue, reducing the effectiveness of the treatment, and the person may not feel anything. If this is the case, immediately modify your technique. This does not mean that the treatment must be painful or uncomfortable, but it should not be more comfortable than the treatment with the small cup.

When you begin using the large cup you should feel the irregularities in the deeper tissue. If it feels smooth, gliding easily over the thighs, SLOW DOWN. The person will begin to see further changes in the leg once the large cup is used because the underlying layers are being altered. The legs may look worse than before, but this is good news and the body is going through an important stage in the process.

Procedure for using the large cup: A greater amount of oil should be applied to the leg when using the large cup, because of the larger circumference of the mouth, which pulls more oil into the cup. Always begin the stroke by placing the mouth of the cup on the skin and waiting at least ½ second to start the stroke. Always move slowly. The success of this technique depends on reaching the deep layers of the connective tissue. If you move too quickly, you will have less effect on the deep congestion and the large cup will not separate the deep tissue, but glide right over it. If it is too comfortable for the person or if the large cup is easy to pull, it means you are going too fast and not reaching deeper layers. It may be necessary initially to use both hands to lift and pull the large cup. If it is too hard to complete a stroke, turn the pressure of the suction down and proceed more slowly.

The face cup: The face cup has the smallest opening and is used to pop the lymph nodes in the neck, and drain the face and neck. It can be used to promote drainage of acne, to release toxins, and during mild colds.

Procedure for the face cup: The face and the neck should be drained very lightly. The face is drained using the drainage stroke just above the jaw line going from the inner neck in an upward direction to the ear. Drain for up to 10 minutes on each side. The neck is drained from the middle of the throat to the ear. The drainage stroke should be extremely light and done for up to 10 minutes on each side. The person should be able to talk easily during this treatment.

### 24 Hour Diet

The sweeping movements of the treatment, running parallel to the lymphatic pathways, guide the lymph, along with its newly acquired fat content, to the nearest lymph nodes. Once through the lymph nodes, the fat-laden lymph passes through the deep lymphatic channels in the abdomen to the thoracic cavity. At this point, the bloodstream becomes high in fat and the fat is used by the body as calories for normal metabolic work. However, if additional fat enters in the day following treatment, it cannot all be used. Some of the fat will revert back to storage and redeposit in other areas. If the 24-hour fat-free diet is not followed, the treatments become ineffective. For best results, a low-fat diet should begin three hours prior to each treatment.

### Application in Liposuction:

The present method of treating the body is very effective prior to Liposuction. Liposuction mainly attacks the middle layer of the fat, sucking out fat cells but it leaves the top layer of the skin dimpled and irregular. Celluderm works subcutaneously to attack the top layer of the skin, breaking up the congestion that contributes to the sluggish lymphatic flow.

Pre-operative: The present method will stimulate the lymphatic system and break apart adhesions that cause congestion. This will hence make it easier for a physician to perform the surgery leading to smoother results.

Post-operative: (1) The present method increases lymph drainage releasing water retained almost instantaneously. Fluid is infiltrated into the body during liposuction. Not all this fluid is released after the procedure and it stays floating around the body. The lymphatic stimulation of the Celluderm releases this fluid so patients can see results immediately. (2) It reduces congestion by breaking up fat clusters subcutaneously that cannot be touched with liposuction, thereby significantly improving results after liposuction. With congestion comes a slower metabolism of fat in the problem areas. They are the hardest area to target because fat is trapped in the subcutaneous layer thereby sticking to the connective tissues. The strength of the vacuum suction causes fat molecules to be released from congested fat cells, pulling the molecules across the cell membrane and releasing them into the lymphatic system. The lymphatic system drains in the thoracic duct, transfers the fat-laden lymph to the bloodstream and taken to the liver to be metabolized. Dramatic results are attained post-op as water is released and fat clusters are diminished.

Lymphatic stimulation is needed in conjunction with liposuction pre-op and post-op to: reduce water retention; speed healing, decrease recovery time, decrease post-op effects by stimulating lymph drainage; reduce irregularities and the bumpy appearance that can occur as a side effect of liposuction; and reshapes the body promoting smoother-looking skin.

### Statistics of Celluderm:

This system is used in a clinical group of 45 persons who have each had a minimum of 10 treatments. If a person has had more than 10 treatments, the results are broken down to reflect 10 treatments. Number of clients that did not show significant changes (less than 5 inches lost in 10 treatments): 5 out of 45 or 1/9 or 10%. In a trial on 40 clients all clients showed significant results on their legs, full and upper body. There was one client with liposuction who lost over 6.25 inches in four different measurements taken around her mid-section for an average of 1.5 inches in each area after just one treatment.

### Body Parts That Can Be Worked On:

The present method can work on any part of the body that has had liposuction done to it. The treatment is a 45 minutes and can be done in 7 different ways. The time spent on each area determines which way to work on a client. 1) Only legs—more time spent on the back of the legs; 2) Only legs—equal time spent on the front and back of the legs; 3) Legs and Upper body—equal time on each area; 4) Legs and Upper body—More time spent on the legs; 5) Legs and Upper body—More time spent on the upper body; 6) Upper body—More time spent on the back and love handles; 7) Upper body—Equal time spent on the back and stomach.

Arms, face, calves, shins and forearms can be added to any treatment but the treatment should not exceed 45 minutes with the exception of extra work on the face.

The treatments use a lot of oil. The carrier oil used may be grape seed oil, but mineral and essential oils can be added as well. Adding mineral oils to the carrier oil makes the oil lighter and easier to clean. Rosemary, cypress, grapefruit, cedar, and lavender are some recommended essential oils.

The client treatment time is 40 minutes. This does not include pre-heating. Full-body drainage can be as long as 45 minutes. If too much fat is loosened, it can redeposit due to overload on the liver.

There is a fine line between the pressure of the suction and the client's threshold of pain. If the suction is too strong, the client cannot relax and the muscles will tighten, closing the lymphatic vessels. The treatment should not be too strong or too weak. The maximum therapeutic results combined with a minimum amount of pain must be determined. Feedback on the client's tolerance is crucial. If the client can tolerate some discomfort while remaining relaxed, the pressure can be increased.

### Celluderm Technology Strokes

Drainage Technique: the basic stroke used to drain fat. Depending on the stage of congestion, one of two strokes is used.

Fast Stroke: a quick stroke along the targeted area that drains the superficial fat. It is best to use this technique when the client is very congested. The small cup is aimed at draining the superficial layers of fat and works best with this stroke.

Slow Stroke: this technique is used with the large cup. It allows fat to be drained at a deeper level. Wait ½ second with the cup on the skin before slowly going over the targeted area. If you go too fast with the large cup, you risk gliding right over the fat.

Friction: a quick back-and-forth movement with the glass cup that is a powerful and effective type of connective tissue manipulation. Start at the inner part of the body and pull the cup back and forth for three to six strokes without releasing the pressure. There is no set direction to follow when using this technique.

Popping: a technique used to release any blockage or obstruction in the lymph nodes. It is similar to using a plunger to unclog the toilet. With the cup on the lymph nodes, hold and release the vacuum hole repeatedly to create the popping action. (You should hear popping sounds.) Repeat over all the lymph nodes in the area.

Massage stroke or follow through: After each pass of the glass cup, use your free hand to follow its path. This not only soothes any irritation, but it also allows you to feel any congestion and determine which areas need extra attention.

Stimulation Technique: This stroke is done on the stomach, following the direction of the colon. Keeping the suction pressure low, gently move the glass cup up and down in a vertical motion for 1-2 seconds in an inverted ‘U’ pattern.

### Treatment Procedure

Procedure or the back of the legs: When working on the legs, avoid the fascia below the saddlebag area because the connective tissue is usually so compacted that it is extremely painful.

1—Turn on equipment and set it to the appropriate strength. 2—Move heating pad from the right thigh onto the left leg. 3—Apply oil liberally to leg before and during each pass. 4—Pop the lymph nodes on the leg two fingers above and below the knee. 5—Use the drainage technique on the right thigh for five minutes. Start at the inner knee and follow along the lymphatic channels. Follow through with the free hand. Check with the client to see if the suction strength needs to be adjusted. Bring the lymph to the drop-off zone at the saddlebag area on the leg. On each passage, bring the lymph to a different area in the drop-off zone in a five-inch arc. 6—Pop the lymph nodes. Use the drainage technique lightly on the right calf for 30 seconds. 7—Pop the lymph nodes. Use the drainage technique on the right thigh for one more minute. 8—If doing 2 passages on the legs, re-wrap the right thigh in the heating pad and follow steps 2-7 on the left thigh and calf. 9—Repeat steps 2-7 on the right leg, but with friction, if possible during the five minute pass. 10—Skipping over the back of the knee, travel up the back of the right leg three times using massage strokes. Wipe off excess oil and wrap the front of the right leg with the heating pad. 11—Repeat steps 2-7 on the left leg, but with friction, if possible, during the five-minute pass. 12—Skipping over the back of the knee, travel up the back of the left leg three times using massage strokes. Wipe off excess oil.

Procedure for back: With full body drainage, the back is done after the first passage of the back of the legs procedure, before the client turns over. With the exception of the love handles, the back treatment should not be uncomfortable. Drainage of the back also releases tension in the back muscles.

1—Heat the right side of the back with the heating pad after finishing the first passage of the right leg. 2—Move heating pad to the left side of the back. 3—Pop lymph nodes along the right side of the spine three times starting from the lower back and moving toward the nape of neck. 4—Keeping the suction pressure low, pull up toward the lymph nodes in the armpit, starting at the iliac crest (upper rear). Repeat, concentrating on the love handles. Begin near the spinal cord moving outward and up. 5—Follow the line of the iliac crest from the sacrum to the outside of the buttocks in a fan-shaped pattern. 6—Work around the scapula in a fan-shaped pattern moving the lymph toward the armpit. 7—Continue this procedure for about seven minutes. 8—Pop lymph nodes along the right side of spine. 9—Remove heating pad from the left side of the back and wrap it around the front of the right leg. 10—Repeat steps 3-8 on the left side of the back. 11—Travel up the back three times starting at the lower back and ending at the shoulders using massage strokes.

Procedure for front of the leg: 1—Remove heating pad from the front of the right leg while client is turning over. Put heating pad on the left leg. 2—Adjust strength of the Celluderm if necessary. 3—Have client raise right knee and use the drainage technique for one minute on outer thigh, hip and saddlebag area with friction, if appropriate. This brings the fat molecules from the back of the legs to the lymph nodes at the groin. 4—To work the inside of the leg, have the client keep the knee bent and allow the leg to fall open. Pop the lymph nodes at the groin and use the drainage technique for one minute on the inner thigh and knee. Begin the pull on the heated adductors from the inside, pulling the cup toward you. This area is very sensitive, so the suction pressure may need adjusting. 5—Pop the lymph nodes at the groin area. 6—Have the client straighten the leg and use the drainage technique on the front of the thigh, outer thigh and knee area for three minutes. Long strokes can be used, going past the lymph nodes to the upper hip. Drain around the knee cap in a diagonal motion with small, light strokes. 7—Travel up the front of the right thigh three times using massage strokes. 8—Remove heating pad from the left leg and place it on the stomach. 9—Repeat steps 3-7 on the left leg. Note: If at any point during the treatment the area you are working on is no longer hot, cut the pass short and reheat the area. The treatment is ineffective when the tissue is cold. This will happen less often as the client has more treatments and circulation improves.

Procedure for the stomach: 1—Remove heating pad from stomach and wrap left arm if arms are going to be treated. 2—Apply oil. 3—Use the stimulation technique along the path of the colon starting at the lower right side of the stomach and moving clockwise in an inverted ‘U’ to the lower left side. Use lower pressure when doing this technique to avoid getting a large amount of flesh stuck on the cup. Repeat. 4—Begin the drainage technique by using light strokes starting at the waist and moving diagonally in a downward motion to just below the belly button for at least one minute. 5—Repeat on the other side. 6—Stimulate the colon once more.

Procedure for arms: Congestion accumulates in the arms in the triceps, and does not generally affect the biceps. 1—Remove heating pad, wrap right arm and apply oil. 2—Rest the left arm on the table at a 90° angle above the head. Strokes are done in a ‘V’ shape starting by the elbow and working toward the armpit. 3—Pop the lymph nodes under the left arm. 4—Pull toward the lymph nodes at the armpit for 30-60 seconds using the small cup. 5—Pop the lymph nodes under the left arm once again. 6—Repeat steps 1-5 on the right arm.

It is to be understood that the present invention is not limited to the sole embodiment described above, but encompasses any and all embodiments within the scope of the following claims.

What is claimed is:

1. A body treatment system comprising:
a. a controllable vacuum pump;
b. a filter to prevent oil, dust and unwanted material to enter the vacuum pump;
c. a microprocessor to control the vacuum level and the vacuum duration;
d. a solenoid valve, which is controlled by the microprocessor;
e. a pressure gauge;
f. plurality of suction cups having different sizes;
g. connection tubings connecting the vacuum pump to the suction cup;
h. said system having plurality of settings, said setting comprising:
(i) Facial setting that is used for facial treatments, having a vacuum range from 0-4 inches of mercury; (ii) Superficial setting that is used for arms, calves, shins and forearms, having a vacuum range from 5-10 inches of mercury; (iii) Deep Drainage setting that is used for legs, back and stomach having a vacuum range from 10-20 inches of mercury; and (iv) Deeper Drainage setting that is used for legs, back and stomach, having a vacuum ranges from 20-25 inches of mercury; and
i. said system having plurality of operating modes, said modes provide vacuum levels for different lengths of time, said modes comprising: (i) Pop mode for opening of lymph nodes having very short time duration of about ½ second duration; (ii) Stimulation mode for stimulating the colon having slightly longer time duration of about 1 second duration; (iii) Drainage mode for drainage strokes having relatively longer time duration of about 2 second duration; (iv) Short Friction mode for friction strokes having longer time durations of about 4 second duration and (v) Long Friction mode for friction strokes having relatively longer time durations of about 6 second duration.

2. The body treatment system of claim 1, wherein said plurality of suction cups comprising of a quick open valve, whereby the vacuum in the cup can be quickly released by a push button on the valve or controlled by said microprocessor.

3. The body treatment system of claim 1, wherein said plurality of suction cups further comprising a roller which provides a light massage after the passage of the vacuum suction cup over a body.

4. A method for treating a body comprising steps of:
a. applying heat on the body part to be treated;
b. providing plurality of different vacuum schemes applied on the body, each of which schemes being distinguished by a set of parameters comprising of a vacuum level, duration of the application of the vacuum, suction cup size used to apply the vacuum on the body, and the rate of stoke of the cup on the body; and
c. a fat free diet after the treatment.

5. A method of claim 4 wherein the diet is kept for 24 hours after the treatment.

6. A method of claim 4 further comprising of a step of consuming Lecithin before the treatment.

7. The method of claim 4, wherein each of said plurality of different schemes comprising a vacuum increasing phase, a main phase, and a vacuum decreasing phase.

8. The method of claim 4, wherein each of said plurality of different schemes comprising vacuum parameters which are dependent on time and/or on vacuum level for the body of each person.

9. The method of claim 4, wherein said plurality of different schemes are obtained from different persons.

10. A method of claim 4 for treating superficial layers of the body, wherein the vacuum suction is applied using a small suction cup, preferably less than 1.5 inches in diameter.

11. A method of claim 4 for treating deeper layers of the body, wherein the vacuum suction is applied using a large cup, preferably no more than 2.0 inches in diameter.

12. A method of claim 4 for treating a face, wherein the vacuum suction is applied using a facial cup, preferably less than ⅞ of an inch in diameter.

13. A method of claim 4 for facial treatment, wherein low levels of vacuum, preferably less than 5 inches of mercury, is used, whereby very light drainage is allowed to reduce water retention and the lymph in the face is stimulated.

14. A method of claim 4 for treatment of superficial layers of the body, wherein medium vacuum levels, preferably between 5-10 inches of mercury, are used.

15. A method of claim 4 for treatment of deeper layers of the body, wherein high vacuum levels, preferably between 10-20 inches of mercury, are used.

16. A method of claim 4 for treatment of deeper layers of the body, wherein high vacuum levels, preferably between 20-25 inches of mercury, are used, whereby faster results are obtained.

17. A method of claim 4, wherein each of said plurality of different schemes comprising of using a small cup and having fast strokes in the beginning, to minimize any pain felt by the person and having the movement in substantially the same direction as the flow of the lymphatic system, and progressively increasing the friction as the client progresses through the treatments,whereby the long drainage strokes of the friction increases circulation to clear the passageway of the lymphatic system for enhanced drainage.

18. A method of claim 4, wherein the method is applied to the area of concern and the motion is from the middle moving toward the outside, starting with a low vacuum setting and gradually increasing the suction during the progression of treatment; continually consulting with the person to determine the ideal pressure, which is defined as the point where the person can feel the pressure, but it is still tolerable, and wherein to start the drainage, beginning with fast strokes in the direction of the lymphatic system and slowing down when the person is ready for deeper drainage.

19. A method of lymphatic stimulation in conjunction with liposuction pre- op and post-op, said method comprising steps of:
a. applying heat on the body part to be treated; and
b. providing plurality of different vacuum schemes applied on the body, each of which schemes being distinguished by a set of parameters comprising of a vacuum level, duration of the application of the vacuum, suction cup size used to apply the vacuum on the body, and the rate of stoke of the cup on the body,
whereby, said method helps to reduce water retention; speed healing, decrease recovery time, decrease post-op effects by stimulating lymph drainage; reduce irregularities and the bumpy appearance that can occur as a side effect of liposuction; and reshapes the body promoting smoother-looking skin,
